(19) United States
(12) Patent Application Publication
(10) Pub. No.: US 2016/0324468 A1
(43) Pub. Date: Nov. 10, 2016

(54) SYSTEM, METHOD AND APPARATUS FOR POST-OPERATIVE BARIATRIC WEIGHT LOSS PERFORMANCE TRACKING

(71) Applicants: Peter H. Kwon, Middletown, NY (US); Gilles Pipernos, West Hartford, CT (US)

(72) Inventors: Peter H. Kwon, Middletown, NY (US); Gilles Pipernos, West Hartford, CT (US)

(21) Appl. No.: 15/148,346

(22) Filed: May 6, 2016

Related U.S. Application Data
(60) Provisional application No. 62/158,328, filed on May 7, 2015.

(51) Int. Cl. A61B 5/00; G01G 19/44; A61F 5/00; H04L 29/08; A61B 5/107

(52) U.S. Cl. CPC A61B 5/4848; H04L 67/12; A61B 5/1072; A61F 5/0013; A61B 5/743; A61B 5/72; G01G 19/44; A61B 2560/0475

(57) ABSTRACT

A mobile application for a mobile computing device that charts a patient's weight loss after a bariatric surgical procedure compared to a reference curve. By having a reference curve for the patient to conduct a self-comparison, the App can potentially encourage the user to try harder to either meet or beat the average weight loss over time, resulting in improved results for the patient.

### CROSS-REFERENCE TO RELATED APPLICATION

This application claims the benefit of priority of U.S. provisional application No. 62/158,328, filed May 7, 2015, the contents of which are herein incorporated by reference.

### BACKGROUND OF THE INVENTION

The present invention relates to bariatrics and, more particularly, to medically monitoring a patient's weight loss following a bariatric surgical procedure.

Current body weight tracking apps allow for user entry of weight over time, but do not give any feedback as to how the user is doing in regards to weight loss. Merely capturing weight values and displaying the weight values do very little in directly motivating the user in modifying the user's diet and behavior, post operatively. While there are many weight-tracking apps available, none provide a reference curve for people having undergone bariatric weight loss surgery.

As can be seen, there is a need for a weight tracking system and method that permits a user, particularly a post operative bariatric patient, to track their weight loss in comparison to observed weight loss averages for similarly situated patients. By having a reference curve for self-comparison, the App can potentially encourage the user to try harder to either meet or beat the average weight loss over time, resulting in improved results for the user.

### SUMMARY OF THE INVENTION

In one aspect of the present invention, a system for comparing bariatric weight loss of a post-operative patient, includes a computer system; and a computer program product comprising a computer-readable, tangible storage device having a non-transitory computer-readable program code stored therein, said computer readable program code containing instructions that are carried out by a processor of the computer system that, when executed, cause the machine to perform the following process steps: 1) receiving a current weight value of the post-operative patient; and 2) presenting the current weight value on a graphical display in comparison to a reference weight loss curve comprising a plurality of historical weight loss values temporally recorded for a plurality of prior bariatric patients.

The step of receiving may further include receiving a manual current weight value via a control of a graphical user interface presented on the display. In other aspects of the invention, the step of receiving may further include automatically receiving the current weight value from a scale in communication with the computer system. The system may also include the step of transmitting the current weight value to a server in communication with the computer system. The current weight value may be stored on the server. In other embodiments, the system includes retrieving the current weight value; and temporally presenting the current weight values as a user weight loss curve. The user weight loss curve may also be presented in a temporal comparison to the reference weight loss curve from a temporal reference point.

In some embodiments, the temporal reference point corresponds to a date a bariatric surgical procedure is performed. In other embodiments, the temporal reference point corresponds to a goal date for achieving a weight loss goal.

In yet other aspects of the invention, the system may also include the step of updating the reference weight loss curve for a subsequent user based on the stored current weight values. The reference curve may also be adjusted for a height and an initial weight of the current post-operative patient. The reference curve may also be generated based on the plurality of historical weight loss values obtained from prior patients having a corresponding type of bariatric surgical procedure as the post-operative patient.

These and other features, aspects and advantages of the present invention will become better understood with reference to the following drawings, description and claims.

### BRIEF DESCRIPTION OF THE DRAWINGS

FIG. 1 is a schematic diagram of an embodiment of a bariatric weight loss tracking and performance system.

FIG. 2 is a flow chart of an embodiment of a bariatric weight loss tracking and performance system.

FIG. 3 is a diagram of a representative system architecture for a bariatric weight loss tracking and performance system.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a system, method, and application for a mobile computing device that permits a post operative bariatric surgical patient to track their weight loss over time and compare their weight loss to observed patient weight loss performance for a population of patients who have undergone a bariatric surgical procedure.

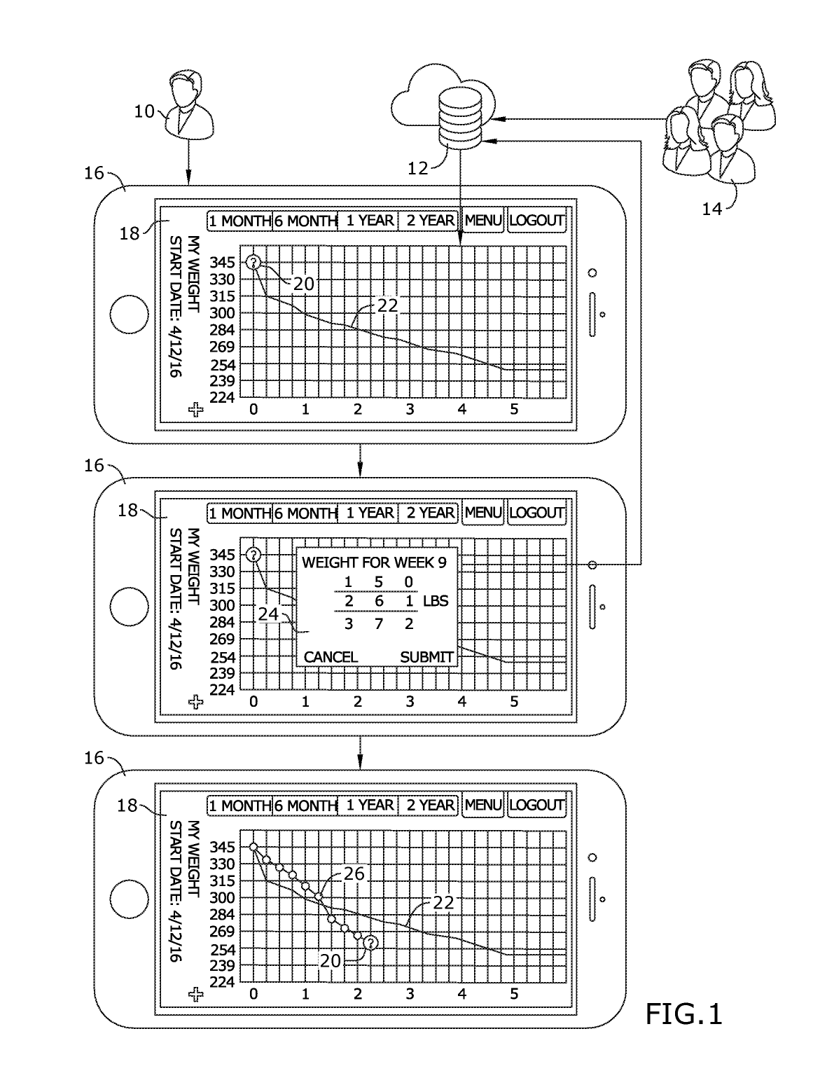

As best seen in reference to FIGS. 1 and 3, a bariatric weight loss tracking and performance system according to embodiments of the present invention includes a user 10, an application server 12, and one or more mobile computing devices 16 in communication with the server 12. The mobile computing device 16 may include a smart phone, a tablet, a laptop computer, and the like.

The mobile computing device 16 is configured with an application program, or app, stored in a non-transitory computer readable memory having computer readable instructions, that when executed by a processor of the mobile computing device 16 present a user interface 18 on a display of the device 16 and send and receive data regarding a patient's post-operative weight loss performance.

The app presents a user interface 18 graphically indicating the patient's 10 recorded and stored measured weight values over a selected time interval. The user interface is also programmed to display a typical weight loss curve 20. The typical weight loss curve 20 is derived from a historical weight loss data record for a plurality of past bariatric surgical patients 14. The application user interface 18 may also be configured fur the user to manually enter a measured weight 24 and to submit the measured weight 24 to the application server 12. In the example shown, the user interface is configured with a scroll wheel display that permits the patient 10 to swipe one of a plurality of scroll wheels to select each digit in their weight. Once the patient 10 has confirmed that the weight is entered correctly, the patient 10 may submit the weight to the server 12 by activation of a submit control icon.

The app may also be configured for obtaining the measured weight 24 of the patient 10 automatically. In this instance the application may permit configuration of a wireless transceiver, such as Bluetooth or Near Field Communications (NFC), of the mobile computing device 16 for communications with an electronic scale (not shown) configured with a compatible communications transceiver. The use of automated measurement may be specified to assist with patient compliance and integrity of the data.

The historical weigh loss data is produced from the observed average weight loss for the former bariatric surgical patients 14. The historical weight loss data may be sorted or organized by bariatric surgical procedure types. The data from, for example a large bariatric surgery program, or a group of participating bariatric surgery programs, may used to create an underlying database to generate the reference curve for each surgery type. The reference curve may also be adjusted for the height and starting weight of the patient. According to other aspects of the invention, the historical data may be supplemented by one or more patients that elect to participate in adding their weight loss performance data to the server 12 and its associated storage devices.

The application user interface 18 may also be configured to graphically display a patient progress chart. The patient progress chart is a graphical representation of the patient's recorded weight values 26 in comparison to a temporal reference point. The user interface may be configurable to display the time reference points in a post operative time, such as days, months, years, measured from the date of the bariatric surgical procedure. Alternatively, the reference points may be configured to a count down to a desired future date, or a target date, for the patient 10 to achieve a post operative weight loss goal.

By constructing a reference curve based upon actual observed average weight loss, adjusted for initial height, weight, and type of surgery, in a single large bariatric surgery practice, the patient can self-monitor his or her weight loss progress against the averages. By having a reference curve for self-comparison, the App can potentially encourage the user to try harder to either meet or beat the average weight loss over time, resulting in improved results for the user.

To make this invention, one must first have the data for the reference curve, then secondly must develop the software that will be able to transform the data for any array of height and initial weight, and the software that will graphically display the reference curve and the entered weights over time in a visually pleasing manner.

The transformation of the data to apply to a specific initial weight for a given height is also desirable. For example, actual initial height and weight data and observed weight loss data from over 5,000 bariatric surgery patients undergoing Lapband placement, sleeve gastrectomy, and gastric bypass was then transformed into initial BMI (Body Mass Index) and observed BMI decreases. Then the reference curve for a person with a specific initial height and weight was generated based on the initial BMI using the transformed data base of BMI decreases observed by surgery type.

If the entered data can be used to modify the reference curve, this would further enhance the App's utility and validity. While this app is being developed for use by surgical weight loss patients, the concept of the reference curve can be used by any number of different non-surgical weight loss programs.

The user 10 can compare his or her weight loss over time against the average reference curve 24. The active use of this app may encourage better compliance with the recommended diet and exercise program, resulting in better weight loss.

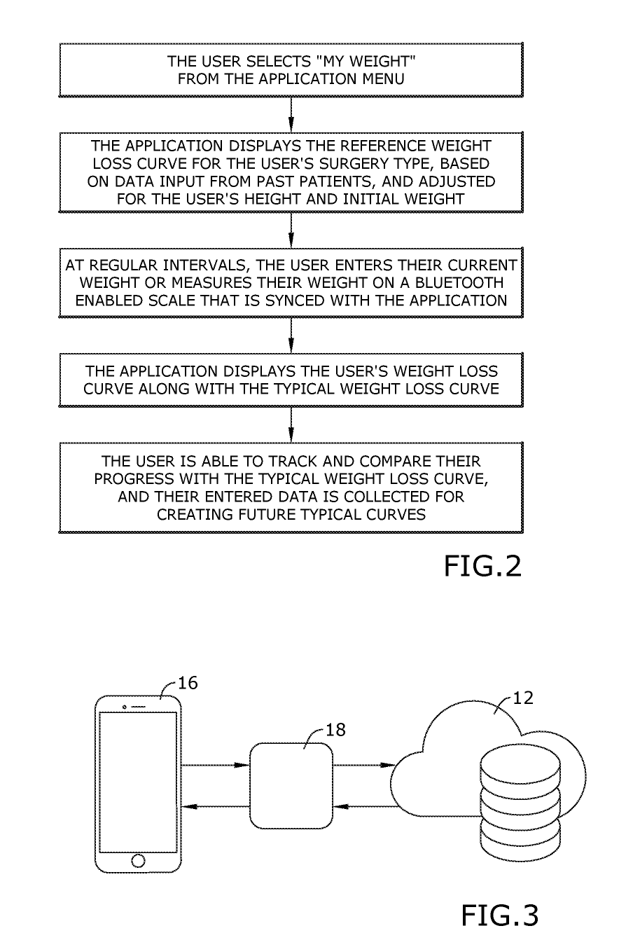

As seen in reference to FIG. 2, the patient, or user 10, may select a “My Weight” menu selection, or control from the application user interface.

After selection of the “My Weight”, the application may then display the reference weight loss curve on the display of the computing device 10. The reference weight loss curve may be configured according to the user's 10 surgical procedure type so that it corresponds from historical patient 14 weight loss performance. The reference weight loss curve may also be adjusted to the user's 10 height and initial or pre operative weight.

The user 10 then enters their current weight 20 at specified intervals. Alternatively, the user 10 may record their current weight 20 automatically, such as via a Bluetooth enabled scale that is synched or paired with the mobile computing device 16.

Once the current weight 20 is entered, the application updates the display to show the user's 10 weight loss curve 26 in comparison with the typical weight loss curve. The user is able to track and compare their progress with the typical weight loss curve, and their entered data is collected to contribute to future reference curves.

By using different or multiple sets of reference data, the app can be used for self tracking in comparison to a particular program's average reference curve and for comparing different weight loss programs, such as a diet or exercise regimen.

The system of the present invention may include at least one computer 16 with a user interface 18. The computer 16 may include any computer including, but not limited to, a desktop, laptop, and smart device, such as, a tablet and smart phone. The computer 16 includes a program product including a machine-readable program code for causing, when executed, the computer 16 to perform steps. The program product may include software which may either be loaded onto the computer 16 or accessed by the computer. The loaded software may include an application on a smart device. The software may be accessed by the computer using a web browser. The computer may access the software via the web browser using the internet, extranet, intranet, host server, internet cloud and the like. 12

The computer-based data processing system and method described above is for purposes of example only, and may be implemented in any type of computer system or programming or processing environment, or in a computer program, alone or in conjunction with hardware. The present invention may also be implemented in software stored on a non transitory computer-readable medium and executed as a computer program on a general purpose or special purpose computer. For clarity, only those aspects of the system germane to the invention are described, and product details well known in the art are omitted.

For the same reason, the computer hardware is not described in further detail. It should thus be understood that the invention is not limited to any specific computer language, program, or computer. It is further contemplated that the present invention may be run on a stand-alone computer system, or may be run from a server computer system 12 that can be accessed by a plurality of client computer systems 16 interconnected over an intranet network, or that is accessible to clients over the Internet.

It should be understood, of course, that the foregoing relates to exemplary embodiments of the invention and that modifications may be made without departing from the spirit and scope of the invention as set forth in the following claims.

What is claimed is:

1. A system for comparing bariatric weight loss of a post operative patient, comprising:
a computer system; and
a computer program product comprising a computer-readable, tangible storage device having a non-transitory computer-readable program code stored therein, said computer readable program code containing instructions that are carried out by a processor of the computer system that, when executed, cause the machine to perform the following process steps:
receiving a current weight value of the post operative patient;
presenting the current weight value on a graphical display in comparison to a reference weight loss curve comprising a plurality of historical weight loss values temporally recorded for a plurality of prior bariatric patients.

2. The system of claim 1, wherein the step of receiving further comprises:
receiving a manual current weight value via a control of a graphical user interface presented on the display.

3. The system of claim 1, wherein the step of receiving further comprises:
automatically receiving the current weight value from a scale in communication with the computer system.

4. The system of claim 1, further comprising the step of:
transmitting the current weight value to a server in communication with the computer system.

5. The system of claim 4, further comprising:
storing the current weight value on the server.

6. The system of claim 5, further comprising:
retrieving the current weight value; and
temporally presenting the current weight values as a user weight loss curve.

7. The system of claim 6, further comprising:
presenting the user weight loss curve in a temporal comparison to the reference weight loss curve from a temporal reference point.

8. The system of claim 7, wherein the temporal reference point corresponds to a date a bariatric surgical procedure is performed.

9. The system of claim 7, wherein the temporal reference point corresponds to a goal date for achieving a weight loss goal.

10. The system of claim 6, further comprising:
updating the reference weight loss curve for a subsequent user based on the stored current weight values.

11. The system of claim 7, wherein the reference curve is adjusted for a height and an initial weight of the post operative patient

12. The system of claim 11, wherein the reference curve is generated based on the plurality of historical weight loss values obtained from prior patients having a corresponding type of bariatric surgical procedure as the post operative patient.
